SEQUENCE LISTING (11 sequences)

SEQ ID NO: 1 - protein, 27 aa, artificial
  Proteins derived from combinatorial library
  MISC_FEATURE (1)..(1): Xaa is A, N or R
  MISC_FEATURE (5)..(5): Xaa is A, R, D
  MISC_FEATURE (8)..(8): Xaa is R, V or P
  MISC_FEATURE (10)..(10): Xaa is V, L or S
  MISC_FEATURE (11)..(11): Xaa is T, G or R
  MISC_FEATURE (13)..(13): Xaa is G or T
  MISC_FEATURE (14)..(14): Xaa is L or A
  MISC_FEATURE (18)..(18): Xaa is V or I
  MISC_FEATURE (21)..(21): Xaa is G, A or R
  MISC_FEATURE (25)..(25): Xaa is G, A or R
XYKNXINXAXXVXXVKRXIDXILAXLP

SEQ ID NO: 2 - protein, 19 aa, artificial
  alpha-helical sequence
LAEAKVLANRELDKYGVSD

SEQ ID NO: 3 - protein, 27 aa, artificial
  specific binding peptide
AYKNAINRAVTVGLVKRVIDGILARLP

SEQ ID NO: 4 - protein, 27 aa, artificial
  specific binding peptide
NYKNRINVALGGTAVKRIIDAILAALP

SEQ ID NO: 5 - protein, 27 aa, artificial
  specific binding peptide
RYKNDINPASRVGAVKRVIDRILAGLP

SEQ ID NO: 6 - protein, 46 aa, artificial
  specific binding peptide
LAEAKVLANRELDKYGVSDAYKNAINRAVTVGLVKRVIDGILARLP

SEQ ID NO: 7 - protein, 46 aa, artificial
  specific binding peptide
LAEAKVLANRELDKYGVSDNYKNRINVALGGTAVKRIIDAILAALP

SEQ ID NO: 8 - protein, 46 aa, artificial
  specific binding peptide
LAEAKVLANRELDKYGVSDRYKNDINPASRVGAVKRVIDRILAGLP

SEQ ID NO: 9 - DNA, 39 bp, artificial
  primer
attaggatccccgtcaggggccgatatcaataactatgc

SEQ ID NO: 10 - DNA, 81 bp, artificial
  primer
tttccgctcgagctattcgtgccattcgattttctgagcctcgaagatgtcgttcaggcccggtttgaagtccaatggcgc

SEQ ID NO: 11 - protein, 27 aa, artificial
  part of ABD wild type sequence
YYKNLINNAKTVEGVKALIDEILAALP

(19) United States
(12) Patent Application Publication
(10) Pub. No.: US 2022/0402978 A1
(43) Pub. Date: Dec. 22, 2022

(54) POLYPEPTIDES MIMICKING EPITOPE OF BROADLY NEUTRALIZING ANTIBODY VRC01 AS ANTIGENS FOR A VACCINE PREVENTING HIV-1 INFECTION

(71) Applicant: UNIVERZITA PALACKEHO V OLOMOUCI, Olomouc (CZ)

(72) Inventors: Petr MALY, Praha 4 (CZ); Milan RASKA, Olomouc (CZ); Milan KUCHAR, Sobulky (CZ); Petr KOSZTYU, Olomouc (CZ); Jaroslav TURANEK, Brno (CZ); Hana PETROKOVA, Praha 10 (CZ)

(73) Assignee: UNIVERZITA PALACKEHO V OLOMOUCI, Olomouc (CZ)

(21) Appl. No.: 17/642,131

(22) PCT Filed: Sep. 8, 2020
(86) PCT No.: PCT/CZ2020/050066

§ 371 Date: Mar. 10, 2022

(30) Foreign Application Priority Data
Sep. 13, 2019 (CZ) PV 2019-585

(51) Int. Cl. C07K 14/16; A61P 31/18; A61K 39/21; C12N 15/113

(52) U.S. Cl. CPC C07K 14/162; A61P 31/18; A61K 39/21; C12N 15/1132; A61K 2039/53

(57) ABSTRACT

A polypeptide mimicking epitope of glycoprotein gp120 of HIV-1 virus that is recognized by a paratope of broadly neutralizing antibody VRC01 and has the length up to 100 amino acid residues and contains an amino acid sequence:

(SEQ ID NO. 1):
X¹YKNX²INX³AX⁴X⁵VX⁶X⁷VKRX⁸IDX⁹ILAX¹⁰LP

X¹ is selected from amino acids A, N, R;
X² is selected from amino acids A, R, D;
X³ is selected from amino acids R, V, P;
X⁴ is selected from amino acids V, L, S;
X⁵ is selected from amino acids T, G, R;
X⁶ is selected from amino acids G, T;
X⁷ is selected from amino acids L, A;
X⁸ is selected from amino acids V, I;
X⁹ is selected from amino acids G, A, R;
X¹⁰ is selected from amino acids R, A, G;
   with a directly attached alpha-helical structure at the N-terminus or C-terminus is disclosed.

### FIELD OF ART

The present invention relates to a novel class of polypeptides that are suitable as antigens mimicking epitope of spike HIV-1 gp120 glycoprotein recognized by broadly neutralizing antibody VRC01, and thus are suitable as immunogens for stimulation of production of HIV-1-neutralizing antibodies and for development of vaccine preventing HIV infection.

### BACKGROUND ART

HIV-1 infection and Acquired Immunodeficiency Syndrome (AIDS) represent global pandemic resulting in an estimated 35 million deaths worldwide. Irrespective of intensive research, no commercial vaccine is available. The most important obstacles are an enormous HIV-1 antigenic variability and unique biochemical, biological, and immunological properties of the most promising vaccine candidate, HIV-1 envelope (Env) glycoprotein which is responsible for HIV-1 attachment to and the entry into the host cell (Robinson H L HIV/AIDS Vaccines: 2018. Clin Pharmacol Ther 2018, 104: 1062-73; Moore P L The Neutralizing Antibody Response to the HIV-1 Env Protein. Curr HIV Res 2018, 16: 21-8). Env is a trimer of gp160 proteins cleaved into two functional subunits: the gp41 trans-membrane glycoprotein and virus surface-exposed gp120 glycoprotein. Majority of identified and cloned human antibodies, able to neutralize broad range of HIV-1 Env variants (bn-mAb) including VRC01, recognize gp120 subunits.

Novel strategies in HIV-1 vaccine development were encouraged after identification of several of such bn-mAbs, because they could limit viraemia, as shown for elite neutralizers, group of individuals with broad and potent neutralizing activity. The generation of HIV-1-specific bn-mAbs under natural conditions is a long-term process lasting for years and difficult to be elicited by conventional vaccination. The majority of identified bn-mAbs exhibit unique properties including a long HCDR3, extraordinary frequencies of V(D)J mutations and poly- or autoreactivity with human lipids and proteins, which seem to be a crucial obstacle for the development of a successful vaccination strategy.

Despite increasing knowledge of molecular structure of Env glycoprotein of HIV-1, its interaction with neutralizing antibodies and mechanism of immune response development, current vaccines induce immune response with low efficiency and insufficient breadth of HIV-1 variants. (T. Q. Zhou, I. Georgiev, X. L. Wu, Z. Y. Yang, K. F. Dai, A. Finzi, Y. D. Kwon, J. F. Scheid, W. Shi, L. Xu, Y. P. Yang, J. A. Zhu, M. C. Nussenzweig, J. Sodroski, L. Shapiro, G. J. Nabel, J. R. Mascola, P. D. Kwong, Structural Basis for Broad and Potent Neutralization of HIV-1 by Antibody VRC01. Science 329, 811-817 (2010); K. J. Bar, M. C. Sneller, L. J. Harrison, J. S. Justement, E. T. Overton, M. E. Petrone, D. B. Salantes, C. A. Seamon, B. Scheinfeld, R. W. Kwan, G. H. Learn, M. A. Proschan, E. F. Kreider, J. Blazkova, M. Bardsley, E. W. Refsland, M. Messer, K. E. Clarridge, N. B. Tustin, P. J. Madden, K. S. Oden, S. J. O'Dell, B. Jarocki, A. R. Shiakolas, R. L. Tressler, N. A. Doria-Rose, R. T. Bailer, J. E. Ledgerwood, E. V. Capparelli, R. M. Lynch, B. S. Graham, S. Moir, R. A. Koup, J. R. Mascola, J. A. Hoxie, A. S. Fauci, P. Tebas, T. W. Chun, Effect of HIV Antibody VRC01 on Viral Rebound after Treatment Interruption. New England Journal of Medicine 375, 2037-2050 (2016)).

One of innovative solutions to overcome current problems with development of efficient vaccination strategy against infection by HIV is to stimulate production of neutralizing antibodies targeting Env glycoprotein of HIV-1 virus by method of directed evolution of proteins that can represent protein replicas of epitopes recognized by well-characterized neutralizing antibodies (bn-mAb). These small binding proteins can be then used as recombinant antigens for the construction of a vaccine, which will stimulate immune system of the host to produce serum antibodies of required specificity and neutralizing breadth, analogical to the originally used neutralizing Env-specific monoclonal antibody (bn-mAb).

### DISCLOSURE OF THE INVENTION

The present invention provides polypeptides mimicking the epitope of glycoprotein gp120 HIV-1 virus, recognized by monoclonal antibody VRC01, developed from artificial binding proteins identified by selection from highly complex combinatorial library of protein variants (FIG. 1) derived from parental structure of albumin-binding domain of streptococcal protein G (Ahmad J N, Li J, Biedermannova L, et al Novel high-affinity binders of human interferon gamma derived from albumin-binding domain of protein G. Proteins 2012, 80: 774-89), by the method of ribosome display.

The present invention provides polypeptides mimicking the epitope of glycoprotein gp120 HIV-1 virus, which is recognized by broadly neutralizing antibody VRC01 (i.e., polypeptide antigens). These polypeptides contain an amino acid sequence: X¹YKNX²INX³AX⁴X⁵VX⁶X⁷VKRX⁸IDX⁹ILAX¹⁰LP (SEQ ID NO. 1), with N-terminally or C-terminally linked alpha-helical structure, said alpha-helical structure is preferably sequence LAEAKVLANRELDKYGVSD (SEQ ID NO. 2). The alpha-helical structure is directly attached to SEQ ID NO. 1.
X¹ is selected from amino acids A, N, R;
X² is selected from amino acids A, R, D;
X³ is selected from amino acids R, V, P;
X⁴ is selected from amino acids V, L, S;
X⁵ is selected from amino acids T, G, R;
X⁶ is selected from amino acids G, T;
X⁷ is selected from amino acids L, A;
X⁸ is selected from amino acids V, I;
X⁹ is selected from amino acids G, A, R;
X¹⁰ is selected from amino acids R, A, G.

Preferably, the present invention provides polypeptides containing an amino acid sequence selected from the group comprising:

(SEQ. ID NO. 3)
AYKNAINRAVTVGLVKRVIDGILARLP,
(SEQ. ID NO. 4)
NYKNRINVALGGTAVKRIIDAILAALP,
(SEQ. ID NO. 5)
RYKNDINPASRVGAVKRVIDRILAGLP,

with N-terminally or C-terminally attached alpha-helical structure.

The present invention preferably provides polypeptides containing sequence selected from the group comprising:

(SEQ. ID NO. 6)
LAEAKVLANRELDKYGVSDAYKNAINRAVTVGLVKRVIDGILARLP,
(SEQ. ID NO. 7)
LAEAKVLANRELDKYGVSDNYKNRINVALGGTAVKRIIDAILAALP,
(SEQ. ID NO. 8)
LAEAKVLANRELDKYGVSDRYKNDINPASRVGAVKRVIDRILAGLP.

Within the framework of the present invention it has been found that polypeptides of this invention bind to the entire IgG monoclonal antibody VRC01 as well as to the Fab fragment of the VRC01. The polypeptide can be arbitrarily extended at both sides, for instance to contain up to 100 amino acid residues, preferably up to 80 or up to 70 or up to 60 or up to 50 amino acids. The immune response elicited by the polypeptides can be affected by a combination of an initial immunization dose and further booster doses, in which a truncated version of the particular protein sequence can narrow the immune response and production of antibodies to a part of the cognate protein corresponding to SEQ ID NO. 1.

The present invention further provides a DNA sequence selected from the group comprising complementary DNA coding for the amino acid sequence of the polypeptides of the present invention, and DNA hybridizing with said complementary DNA under conditions of high stringency. Conditions of high stringency refer to the following conditions and solution for washing-off the labeled DNA probe: washing solution containing 0.5×SSC+0.1% SDS, temperature of 60° C.

The present invention further includes the use of said DNA sequence for the preparation of polypeptides or recombinant proteins produced in bacterial, yeast, insect, mammal or human host cells, and also these host cells, containing at least one DNA sequence of the present invention.

The present invention further includes the use of said DNA sequence as an active ingredient of DNA vaccine for prevention of HIV-1 virus infection. DNA vaccines contain DNA to be introduced into cells, so the host cells directly produce antigen stimulating preventative immune response. Immune cells recognize such antigen as heterogeneous structure, mature and are responsible for development of antigen-specific immune response. DNA can be introduced into the host organism freely or encapsulated in a protein to simplify host cell entry.

Polypeptides according to the present invention are suitable for use in pharmaceutical technology, especially as mimicking recombinant protein ligands for development of more efficient vaccine preventing HIV-1 virus infection. To this end, it is particularly preferred to attach further auxiliary proteins to the polypeptides of the present invention, said auxiliary proteins being suitable for stimulation of antibody production. Examples of auxiliary proteins include serum albumin, heat shock protein hsp70 or helical spacer protein TolA or its truncated version TolS. These auxiliary proteins can be covalently linked to polypeptides, thus forming a chimeric protein. Furthermore, the polypeptides of the present invention may be modified by an attachment of auxiliary N- or C-terminal sequences (tags), which allow their specific detection or their oriented immobilization to surface of carriers such as nanoliposomes, resulting in enhancement of immunization efficacy. Such tags include, for instance, affinity or detection tags as poly(His), FLAG, AviTag, HA, Myc, S-tag or V5-tag.

Polypeptides of the present invention, defined by the amino acid sequence shown above, stimulate production of serum antibodies after being used for immunization of experimental animals. Hyperimmune sera of the immunized animals suppressed infection of reporter cells by tested Env-pseudotyped viruses in the model system and this represents one of the key mechanisms of HIV-1 infection control and one of the targets for development of a preventative vaccine which is still not available at the market. The polypeptides were identified from ABD-derived library of randomized peptides as peptides with the highest specific binding to monoclonal antibody VRC01, which was identified in an individual infected by HIV-1 virus as one of the crucial factors maintaining long-lasting low level of HIV-1 in his serum and contributing to resistance against AIDS development even without the use of antiretroviral agents. VRC01 is known by its ability to neutralize a broad spectrum of HIV-1 variant identified in different regions of the world. The developed polypeptides mimic the structure recognized by VRC01 antibody (antibody-recognized epitope). The advantage of these polypeptides, in comparison to the vaccines currently being tested, is their easy preparation, stability and absence of posttranslational modifications, and this enables their easy biotechnological production in prokaryotic host cells Escherichia coli and their further utilization as vaccine antigens.

### BRIEF DESCRIPTION OF DRAWINGS

FIG. 1. Schematic representation of elicitation of Env-specific neutralizing serum antibodies using protein binders selected from ABD library. Broadly neutralizing antibody VRC01 was used as a target for the selection of binders from a combinatorial ABD library with a theoretical complexity 10¹⁴ variants. The negative selection was used to minimize presence of binders not involved in epitope recognition. Positive selection was performed in 96-well plates with immobilized VRC01 bn-mAb, followed by mRNA isolation, reverse transcription to cDNA, and ribosome display selection. After several selection rounds, a library of cDNA variants called VRA binders was introduced into a plasmid vector. Three VRA variants VRA017, VRA019 and VRA177 were identified as the most promising candidates and in the form of recombinant fusion proteins, including a truncated VRA017 version designated VRA017S, were used for the immunization of experimental mice followed by the analysis of their hyperimmune sera concerning their HIV-1 Env-specificity and HIV-1 pseudovirus-neutralizing activity.

FIG. 2. Identification of VRA ligands of VRC01 paratope selected from ABD library. Variants preferentially binding to VRC01 IgG were identified by ELISA. Cell lysates of VRA clones were screened for binding to VRC01 IgG and isotype IgG control. The VRA clones were produced as biotinylated His6-VRA-TolA-AVI fusion proteins and binding to IgG was visualized by the streptavidin-HRP conjugate. Parental His6-ABDwt-TolA-AVI biotinylated protein was used as a negative control. Binding of V5-tag-gp120 glycoprotein to VRC01 antibody was used as a positive control, detected by anti-V5 Ab-HRP conjugate.

FIG. 3. ABD domain structure with 11 randomized amino acids.

FIG. 4a. Binding of VRA017-, VRA019- and VRA177-TolA-AVI proteins to VRC01, isotype control IgG kappa and BSA in ELISA. Binding of ABDwt-TolA-AVI as the parental non-mutated protein to VRC01 is shown in comparison to VRA019-TolA-AVI.

FIG. 4b. Binding of VRA017-, VRA019- and VRA177-TolA-AVI proteins to VRC01/Fab fragment and BSA in ELISA. All the VRA and ABDwt proteins were biotinylated and detected by streptavidin-HRP conjugate. Each experiment is shown as the mean value of triplicate with standard deviation (SD).

FIGS. 5a and 5b. The VRA proteins compete with gp120 for binding to VRC01. Increasing concentration of gp120 inhibits binding of the VRA017-, VRA019-, and VRA177-TolA-AVI proteins at a constant concentration 2×10⁻⁷ M to VRC01 IgG (FIG. 5a) and VRC01 Fab (FIG. 5b). All the VRA and ABDwt proteins were biotinylated and detected by streptavidin-HRP conjugate. Results of each experiment are shown as the mean value of triplicate (duplicate in cases of the VRA019 and VRA177 competition experiments) with standard deviation (SD).

FIG. 6. The small binding protein VRA017 competes with gp120 glycoprotein for binding to VRC01 antibody. Increasing concentration of VRA017-TolA-AVI fusion protein as the competitor reduces binding of gp120 at constant concentration 5×10⁻¹⁰ M to VRC01 IgG (left) and to VRC01 Fab fragment (right). Detected by ELISA with anti-V5 Ab-HRP conjugate.

FIGS. 7 a-c. Sera from mice immunized with VRA017, VRA177, VRA019, and VRA017S specifically recognize HIV-1 Env.

FIG. 7a. Mice were immunized in two independent experiments by the administration of three doses of the particular ABD variant including control wild-type ABD (ABDwt), three VRC01-binding variants VRA017, VRA019 and VRA177, and the truncated version VRA017S. Each individual group consists of 5 animals. Following immunization, sera were collected and Clade B multimerized recombinant Env variants (gp120 MBL), without any purification and identification tags, was used for testing of Env-specific serum antibody titres of IgM, all IgG isotypes (IgGtot), IgG1, and IgG2a by ELISA.

FIG. 7b. Env-specific serum IgGtot from experiment I.

FIG. 7c. Env-specific serum IgGtot, IgG1, IgG2a, and IgM from experiment II. Statistical comparison was performed by ANOVA Kruskal-Wallis test with Dunn's post-test (* P<0.05, ** P<0.01).

FIG. 8a. VRC01 competes with sera of immunized mice for binding to gp120. Plates were coated with gp120. VRC01 serially diluted in blocking buffer (in doublets) was applied with mouse sera diluted 1:400. After washing, the plates were incubated with rabbit HRP-conjugated anti-mouse IgG antibody, washed, developed with the substrate and optical density was measured at 490 nm. Mean values are indicated by horizontal lanes.

FIG. 8b. Naïve serum as well as 10E8 antibody were used as negative controls.

FIGS. 9a-c. Titration curves of pooled mice sera during HIV-1 pseudovirus neutralization assay. Neutralization assays were performed using set of HIV-1 Clade B and C pseudoviruses of Tier 2 or 3 with TZM-bl indicator cells. Serially diluted serum samples in duplicates were incubated with pseudoviruses. The pseudoviruses load was set to achieve approximately 150 000 RLU in 150 μl of DMEM in the absence of sera. After the incubation, TZM-bl cells at a density of 10⁵ cells/ml were added, incubated, lysed, substrate was added and luminescence was measured. FIGS. 9a, 9b and 9c show titration curves of particular pseudoviruses and mice sera of individual immunize groups.

FIG. 10. Analysis of thermal stability of the VRA017S, VRA019S and VRA177S proteins using thermal shift assay (TSA). Normalized thermal melting fluorescence curves of the His6-VRA-TolS-AVI binding proteins (left) and the first derivative of fluorescence versus temperature (right) are shown. The melting point is given as the lowest point of the dashed curve. The measurement was done in duplicates and averaged. Identified temperature melting points (Tₘ) for VRA017S, VRA019S and VRA177S binding proteins correspond to Tₘ 50° C., 50° C. and 58.5° C., respectively. Tₘ for parental non-mutated His6-ABDwt-TolS-AVI fusion protein is 55.5° C. (dotted line).

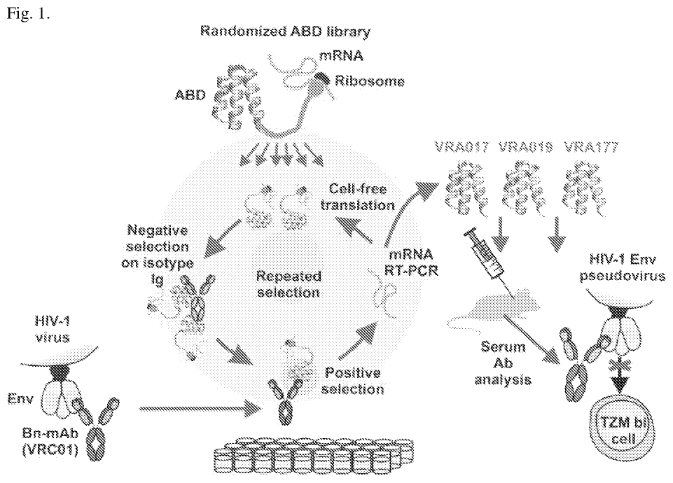

### EXAMPLES OF CARRYING OUT THE INVENTION

### Material and Methods

### Construction and Production of Recombinant Gp120

Multimeric recombinant protein gp120 of “Clade B” with N-terminal His-tag and C-terminal V5-tag was prepared using a protocol described previously (Raska M, Takahashi K, Czernekova L, et al Glycosylation patterns of HIV-1 gp120 depend on the type of expressing cells and affect antibody recognition. The Journal of biological chemistry 2010, 285: 20860-9; Raska M, Moldoveanu Z, Novak J, et al Delivery of DNA HIV-1 vaccine to the liver induces high and long-lasting humoral immune responses. Vaccine 2008, 26: 1541-51; Raska M, Czernekova L, Moldoveanu Z, et al Differential glycosylation of envelope gp120 is associated with differential recognition of HIV-1 by virus-specific antibodies and cell infection. AIDS Res Ther 2014, 11: 23) in concentration 1.2 mg/mL. This protein was used for competition assays with VRA proteins.

### Antibodies Used for Preselection, Selection and Further Characterization

Broadly neutralizing human anti-HIV-1 gp120 monoclonal antibody VRC01 (RRID: AB_2491019) was obtained from Dr. John Mascola (cat #12033) (Wu X, Yang Z-Y, Li Y, et al Rational Design of Envelope Identifies Broadly Neutralizing Human Monoclonal Antibodies to HIV-1. Science 2010, 329: 856-61) through the NIH AIDS Reagent Program, Division of AIDS, NIAID, NIH. Fab fragment of VRC01 was prepared using Fab Micro Preparation kit (Pierce, ThermoFisher Scientific, Waltham, Mass.) according to the manufacturer's instructions. 125 μL of VRC01 antibody (100 μg) was applied onto a column containing equilibrated immobilized papain and incubated for 5 hours at 37° C. The digested antibody was eluted by centrifugation and added onto the column with equilibrated immobilized Protein A. The column was centrifuged to collect Fab fragment. The concentration of purified Fab fragment was measured. VRC01 IgG protein, as well as its Fab, were tested for its activity to bind gp120 when immobilized on Polysorp plate (NUNC, Roskilde, Denmark) in ELISA. Human IgG kappa 1 mg/mL (purified myeloma protein, Sigma-Aldrich, St. Luis, Mo.) was used as a negative control in ELISA as well as an isotype control for preselection in ribosome display, stored as 1 mg/mL source stock at −20° C. VRC01 mAb was used for ribosome display as a target protein, stored as 3 mg/mL source stock in PBS (pH 7.2) at −80° C.

### ABD Library Assembly and Ribosome Display Selection

ABD-derived combinatorial library was assembled by PCR (Ahmad J N, Li J, Biedermannova L, et al Novel high-affinity binders of human interferon gamma derived from albumin-binding domain of protein G. Proteins 2012, 80: 774-89) and used for in vitro translation and further ribosome display selection (Kuchar M, Vankova L, Petrokova H, et al Human interleukin-23 receptor antagonists derived from an albumin-binding domain scaffold inhibit IL-23-dependent ex vivo expansion of IL-17-producing T-cells. Proteins 2014, 82: 975-89). Three- and five-round RD selections were performed, 96-well Polysorp plates (NUNC) were coated by VRC01 IgG diluted in coating 100 mM bicarbonate/carbonate solution (pH 9.6) at a concentration according to the adjusted stringency in each round of ribosome display selection procedure: 1st round—50 μg/mL, 2nd round—25 μg/mL, 3rd round—10 μg/mL, 4th round—5 μg/mL and 5th round—5 μg/mL. Preselection procedure was performed in wells coated with human IgG1 kappa antibody (Sigma-Aldrich) at a constant concentration of 25 μg/mL in each round. Final cDNA after the third and fifth round of the selection was amplified by PCR and introduced into a pET-28b vector carrying cloned tolA-AviTag sequence (Krizova L, Kuchar M, Petrokova H, et al p19-targeted ABD-derived protein variants inhibit IL-23 binding and exert suppressive control over IL-23-stimulated expansion of primary human IL-17+ T-cells. Autoimmunity 2017, 50: 102-13) and introduced into E. coli XL1 blue host cells.

### Production of ABD-Derived Protein Variants

Protein variants were produced in the form of fusion recombinant proteins His6-VRA-TolA-AVI allowing in vivo biotinylation of the binding proteins at C-terminus (Krizova L, Kuchar M, Petrokova H, et al. p19-targeted ABD-derived protein variants inhibit IL-23 binding and exert suppressive control over IL-23-stimulated expansion of primary human IL-17+ T-cells. Autoimmunity 2017, 50: 102-13). Truncated fusion VRA proteins were constructed by replacement of full-length tolA (UniProt accession number: P19934, NCBI Reference Sequence: NC_000913.3) with its C-terminal part called tolS via PCR with primers tolA-C-end 5′-ATTAGGATCCCCGTCAGGGGCCGATATCAATAACTATGC-3′ (SEQ ID NO. 9) and tolA-AVI_rev1 5′-TTTCCGCTCGAGCTATTCGTGCCATTCGATTTTCTGAGCCTCGAAGATGTCGTTCAGG CCCGGTTTGAAGTCCAATGGCGC-3′ (SEQ ID NO. 10). VRA protein binders were produced as in vivo biotinylated proteins in E. coli BL21 (DE3) BirA strain with added 50 μM d-biotin (prepared 5 mM solution in 10 mM bicine buffer, pH 8.3) in LB medium containing kanamycin (60 μg/mL) and chloramphenicol (30 μg/mL). Protein production was induced at 35° C. by 1.5 mM IPTG after the culture reached the density OD₆₀₀=0.6. Cells were harvested in 4 hours after induction, sonicated in TN buffer (50 mM Tris, 150 mM NaCl, pH 8.0), centrifuged (40 000×g, 20 min, 4° C.) and subsequently, bacterial lysates were analyzed or proteins were purified on Ni-NTA agarose column.

### Screening of ABD Variants by ELISA

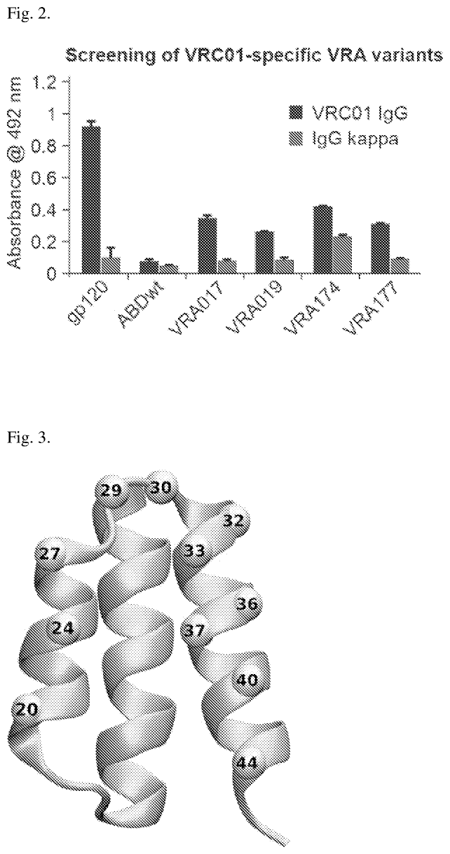

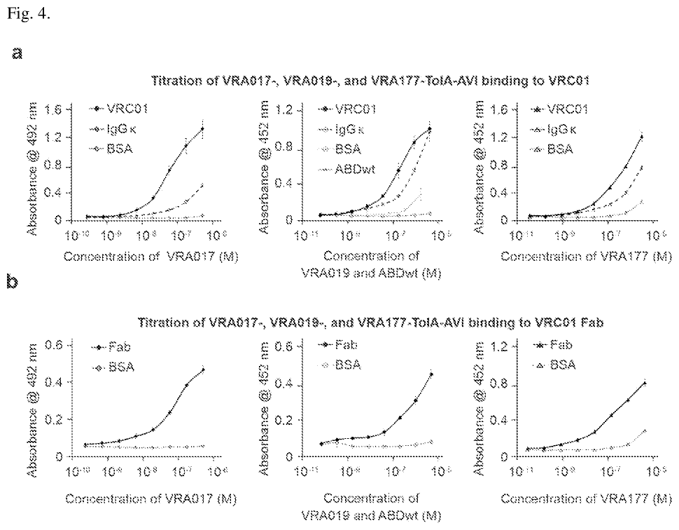

Cell lysates of clones of E. coli BL21 BirA host cells producing biotinylated protein variants were prepared using lysozyme solution (PBS buffer, 0.05% Tween, 1% lysozyme, 25 U/mL benzonase, pH 7.4) or using sonicator (Misonix 3000). Polysorp plate (NUNC, Roskilde, Denmark) was coated with VRC01 IgG1 (5 μg/mL) or IgG1 kappa (5 μg/mL) in coating buffer (100 mM bicarbonate/carbonate buffer, pH 9.6) at 7° C. overnight. Next day, the plate was washed by PBST solution (PBS buffer containing 0.05% Tween, pH 7.4) and wells were blocked by PBSTB (PBS buffer pH 7.4, containing 0.05% Tween and 1% BSA). The lysate samples (diluted 33×), purified protein variants as well as ABDwt negative control diluted in PBSTB were applied and their binding was detected using streptavidin Poly-HRP conjugate (Pierce) diluted in PBSTB 1:10 000. The V5-tagged gp120 recombinant protein was diluted in PBSTB and detected by anti-V5 tag-HRP conjugate in PBSTB (1:10 000). Protein binding was visualized by the enzymatic reaction of HRP with OPD substrate (Sigma-Aldrich, St. Luis, Mo., USA) in citrate buffer (3.31% sodium citrate tribasic dihydrate, phosphoric acid, pH 5.0), or TMB-Complete 2 substrate (TestLine Clinical Diagnostics s.r.o., Brno, Czech Republic) and the reaction was stopped by 2 M sulphuric acid, and the absorbance was measured at 492 or 450 nm, respectively. Using this method, almost 800 lysates of bacterial clones were screened and four variants were found, which preferentially bind to broadly neutralizing antibody VRC01 in comparison to control isotype antibody IgG kappa (FIG. 2). Those binding proteins were designated as VRA proteins. The variants VRA017 (SEQ ID NO. 6) and VRA019 (SEQ ID NO. 7) were obtained in five-round ribosome display (RD) selection, while VRA174 and VRA177 variants were found in three-round RD selection. DNA sequence analysis revealed the sequence identity of VRA174 and VRA177 variants and, therefore, only the clone VRA177 (SEQ ID NO. 8) (Table 1, FIG. 3) was used for further analysis. This method was further used for the verification of specific binding of VRA017, VRA019 and VRA177 proteins to the immobilized neutralizing antibody VRC01 (FIG. 4a) as well as to immobilized Fab fragment of the VRC01 IgG (FIG. 4b) in comparison to the binding to isotype control IgG kappa.

### Competition ELISA

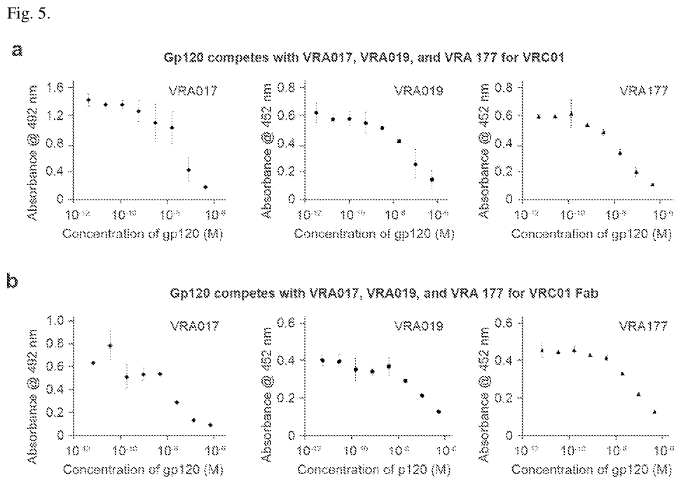

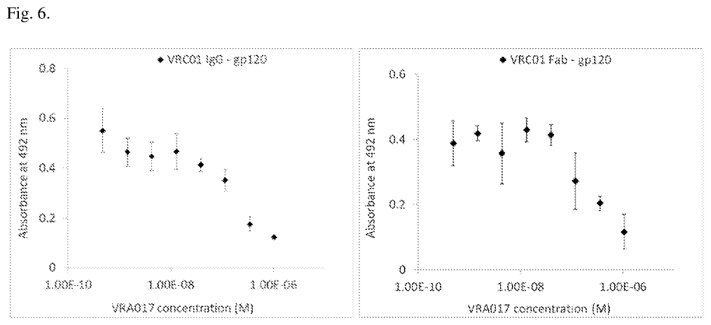

The wells of Polysorp plate (NUNC, Denmark) were coated with VRC01 IgG antibody or Fab fragment, diluted in coating buffer (5 μg/mL). The coted wells were blocked with PBSTB solution and V5-tagged gp120 was applied as a serially diluted competitor in PBSTB solution containing VRA protein variant at constant concentration (5 μg/mL) and binding of the in vivo biotinylated VRA017, VRA019 and VRA177 (as His6-VRA-TolA-AVI fusion protein) was detected by streptavidin-HRP conjugate. Alternatively, VRA017 protein was applied as a serially diluted competitor in PBSTB at the constant concentration of V5-tagged gp120 (1.2 μg/mL) and detection of the bound gp120 was performed using anti-V5 tag-HRP antibody conjugate. Results were visualized by the enzymatic reaction of HRP with OPD substrate (Sigma-Aldrich, MO) in citrate buffer, or TMB-Complete 2 substrate (TestLine Clinical Diagnostics s.r.o., Brno) and the reaction was stopped by 2 M sulfuric acid and absorbance at 492 or 450 nm was measured, respectively. This method was used to demonstrate results in which increasing amount of glycoprotein gp120 inhibits binding of VRA017, VRA019 and VRA177 to immobilized VRC01 IgG as well as to Fab fragment prepared by cleavage of VRC01 IgG (FIG. 5b). These results support the hypothesis that protein variants VRA017, VRA019 and VRA177 recognize variable region of antibody VRC01, so they can be recognized as a non-cognate epitope of the VRC01. Moreover, the binding competition between VRA017 and gp120 was also supported by another experiment confirming that an increasing concentration of VRA017 results in the inhibition of glycoprotein gp120 binding to immobilized VRC01 IgG as well as to its Fab fragment (FIG. 6).

### Sequence Analysis of Selected Variants

Plasmid DNA coding for full-length protein variants was sequenced (Core facility—Genomics, Faculty of Science, Charles University, BIOCEV, Vestec). Multiple alignments of amino acid sequences were performed using NCBI BLAST (National Center for Biotechnology Information, https://www.ncbi.nlm.nih.gov/). Several dozen selected clones were analyzed and the most important ones are shown in Table 1.

TABLE 1
Multiple alignment of amino acid sequences of VRA binding proteins
and parental ABDwt. Bold text indicates 11 positions in which ABD
amino acids in the 20-46 region were randomized.

| | 20 24 27 2930 3233 3637 40 44 |
|---|---|
| ABDwt | Y Y K N L I N N A K T V E G V K A L I D E I L A A L P |
| VRA017 | A Y K N A I N R A V T V G L V K R V I D G I L A R L P |
| VRA019 | N Y K N R I N V A L G G T A V K R I I D A I L A A L P |
| VRA174 | R Y K N D I N P A S R V G A V K R V I D R I L A G L P |
| VRA177 | R Y K N D I N P A S R V G A V K R V I D R I L A G L P |

### Immunization of Experimental Mice

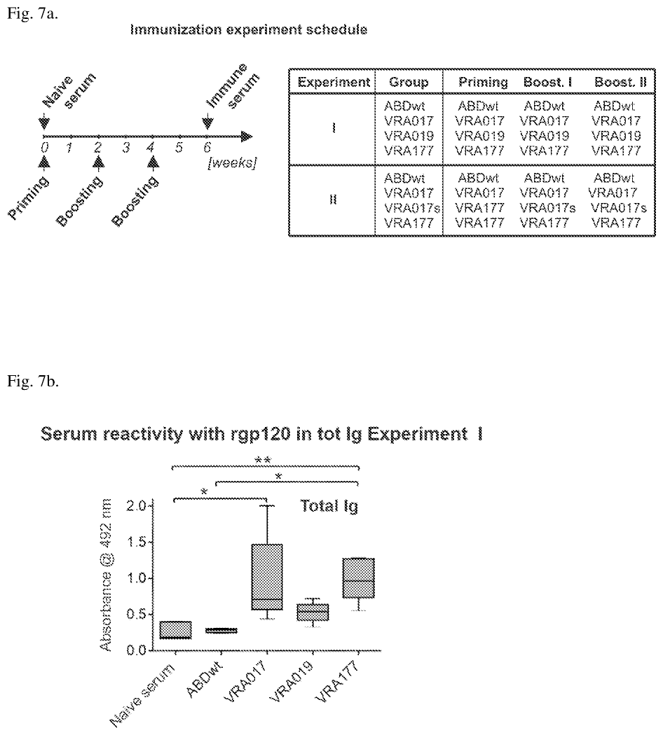

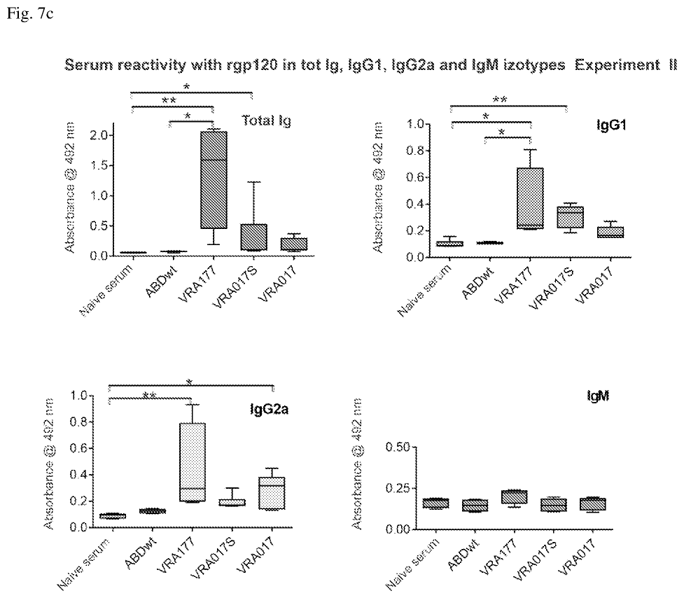

All experiments were performed on 6-to 8-weeks old female BALB/c mice purchased from AnLab (Brno, Czech Republic) under standard housing conditions according to ARRIVE guidelines. The vaccination experiments were approved by the Ethics Committee of the Faculty of Medicine and Dentistry (Palacky University Olomouc, Czech Republic), and the Ministry of Education, Youth and Sports, Czech Republic (MSMT-15434/2015-7). Preimmune serum samples (130 μl per animal) were obtained using tail vein blood sample collection approach. Each mouse was immunized three times with the corresponding ABD/VRA variant. Two independent immunization experiments were performed. The first experiment used VRA017, VRA019, and VRA177 variants and ABD wild type (ABDwt) for the evaluation of immunogenicity and specificity of elicited murine serum antibodies. Results are presented in FIG. 7a,b. The second experiment (FIG. 7a,c) tested VRA017, VRA177, and a truncated version of the VRA017 protein designated as VRA017S with a truncated C-terminal TolS-AVI segment that allows to narrow the focus of the immune response to the VRC01-like epitope or to ABDwt control. All immunizations were performed by intradermal route with equal doses 20 μg of ABD/VRA variant (diluted in 50 μl of sterile PBS) per mouse per one immunization mixed 1:1 (v:v) with Freund adjuvant. Our results confirmed that immunization of mice with VRA proteins lead to the elicitation of antibodies against tested variants of VRA proteins and that the sera of mice immunized with VRA variants targeted gp120 significantly in comparison to the control sera of non-immunized animals or individuals immunized with the control ABD protein. This shows that the surface of the individual VRA variants exhibits a substantial shape complementarity to paratope of the neutralizing antibody VRC01 and, thus, could mimic a part of HIV-1 Env glycoprotein.

### Env-Specific Serum Antibody Determination by ELISA

To determine the reactivity of mice sera with HIV-1 Env, a recombinant trimeric Clade B gp120 MBL lacking detection or purification tags was used in ELISA. Maxisorp plates (NUNC, Roskilde, Denmark) were coated with gp120 MBL (50 ng/well) overnight at 4° C. Plates were washed and blocked with 1% BSA/PBS/Tween20 for 3 hours at room temperature. Sera were serially diluted (starting from dilution 1:100) in blocking buffer (in duplicates) and incubated overnight at 4° C., to identify a single dilution corresponding to the linear proportion of titration curves obtained from the majority of VRA-immunized animals used in the final comparison. Final serum dilution was set to 1:400. To detect the bound antibodies specific to gp120, plates were washed and incubated with rabbit anti-mouse IgG, IgG1, IgG2a, and IgM secondary antibody conjugated with horseradish peroxidase (Sigma-Aldrich, St. Luis, Mo., USA) diluted in blocking buffer for 3 hours at room temperature. Signal was developed with O-phenylenediamine-H₂O₂ substrate. The reaction was stopped with 1 M sulphuric acid and the absorbance was measured at 492 nm. This methodical procedure was used to obtain results shown in FIG. 7c. Presented data demonstrate that sera of mice immunized with VRA017 and VRA177 proteins have a significantly increased binding to trimeric version of recombinant glycoprotein gp120 for IgG1, IgG2a and total IgG, while IgM antibodies do not bind to the gp120.

Competition of VRC01 with Hyperimmune Mice Sera for Gp120 Binding Tested by ELISA

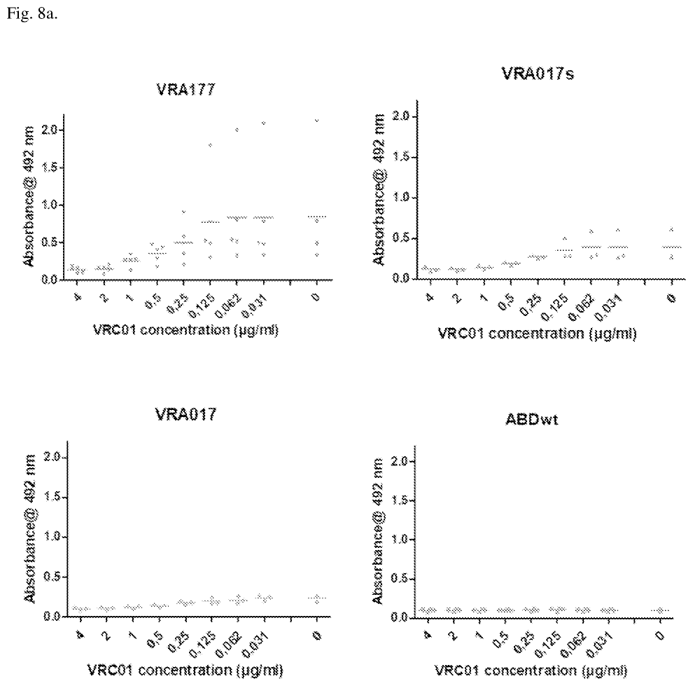

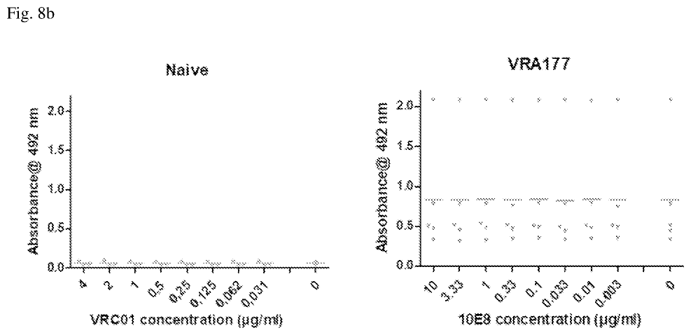

Plates were coated as described above for Env-specific serum antibody determination by ELISA. VRC01 serially diluted in blocking buffer (in doublets) was applied with mouse sera diluted 1:400. To detect bound murine antibodies, plates were washed and incubated with rabbit anti-mouse IgG secondary antibody conjugated with horseradish peroxidase diluted in blocking buffer for 3 hours at room temperature. Plates were developed and measured as mentioned above. This experimental procedure was performed to obtain results shown on FIG. 8a. These results show that the increasing concentration of neutralization antibody VRC01 blocks the binding of diluted sera of mice immunized with VRA177, VRA017S and VRA017 to immobilized gp120. This confirms the competition of monoclonal antibody VRC01 with newly produced serum anti-VRA antibodies and underscores the specificity of newly produced serum antibodies against HIV-1 gp120. This finding is further supported by the inability of control gp120-irrelevant anti-Env antibody 10E8 to compete for binding with sera of immunized mice (FIG. 8b).

### Virus Neutralization Assay

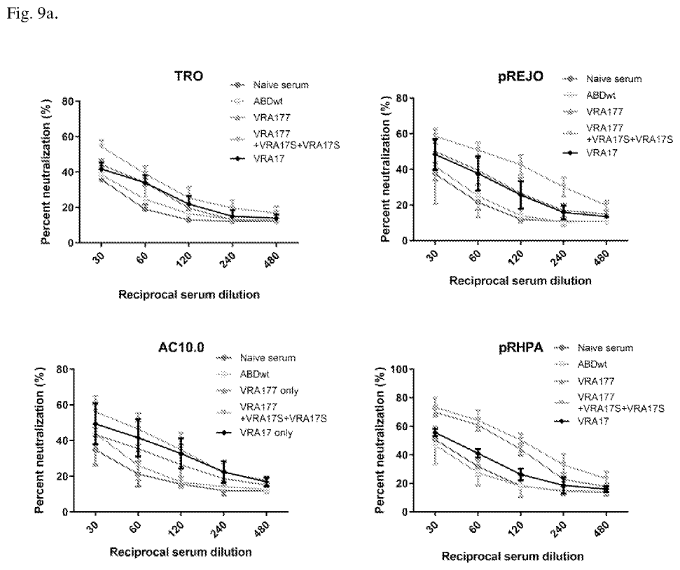

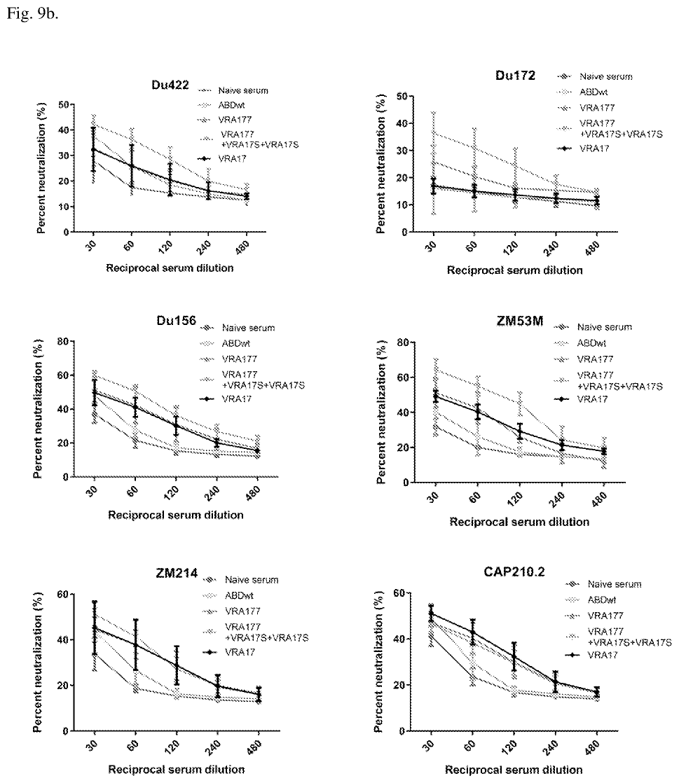

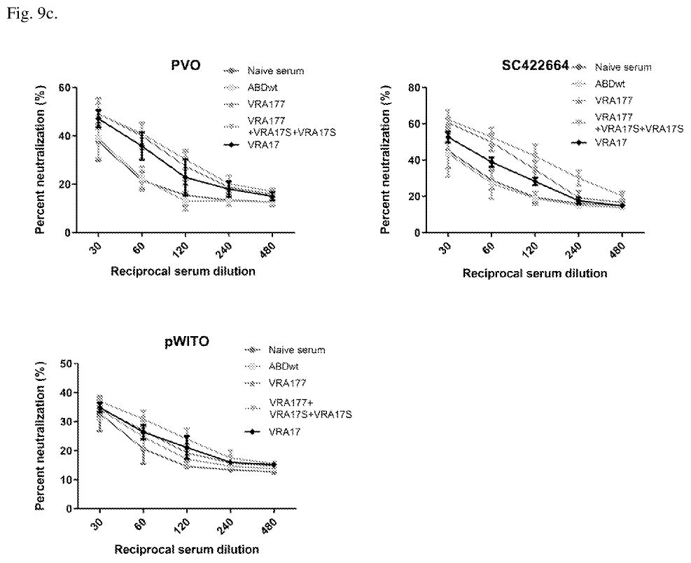

Neutralization assay was performed using various pseudoviruses from clade B and clade C produced in HEK293/17 cell line. Cells at 60-90% confluency in 75 cm² culture flask were co-transfected using transfection reagent FuGene6 (Promega, Madison, Wis.). Before transfection, 8 μg of plasmid pSG3deltaEnv, 4 μg of plasmid encoding Env and 48 μl of FuGene6 were mixed with DMEM culture medium in a total volume of 800 μl and incubated 30 minutes at room temperature. Then, the mixture was added to 12 ml of RPMI-1640 in a flask with cells. After 2 days, culture medium with produced pseudoviruses was harvested, aliquoted and stored at −80° C. until used. Neutralization assay was performed using TZM-bl cell line stably expressing CD4 receptor, CCR5, and CXCR4 co-receptors and containing genes for luciferase and β-galactosidase under the control of HIV-1 long-terminal-repeat promotor (NIH AIDS Research and Reference Reagent Program, Division of AIDS, NIAID, NIH). Serially diluted serum samples in duplicates were incubated with pseudoviruses at approximately 150 000 RLU in 150 μl of DMEM. After 90 minutes' incubation at 37° C., 100 μl of cells at a density of 10⁵ cells/ml was added. The plate was incubated at 37° C. in 5% CO2 atmosphere for 48 hours. Then, 150 μl of culture medium was removed and 100 μL of lysis buffer containing luciferin (Promega) was added. After 2 minutes, 100 μL of lysed cells were transferred into black 96-well plates and luminescence was measured using HP luminometer. This methodical procedure was used for obtaining of a set of neutralization results summarized in Table 2. Our results show that sera of mice immunized with VRA177 and VRA017 proteins exhibit a neutralization activity, which is presented on the set of 13 prepared HIV pseudoviruses. Mice sera of VRA177 variant blocked the binding of 5 types of pseudoviruses to indicator human cells, while sera of mice immunized with VRA017 blocked binding of 2 types of pseudoviruses only. The best neutralizing activity was achieved by a combinatory immunization with VRA177TolA and VRA017S eliciting high titers of serum antibodies blocking the binding of 8 of 13 tested pseudoviruses. Detailed binding curves of hyper immune and naïve mice sera for each type of pseudovirus are shown on FIG. 9a,b,c. Summarized combinatory data demonstrate, that the produced binding proteins VRA177, VRA017 and VRA017S have the ability to mimic epitope of the neutralizing antibody VRC01 as their use for the immunization of animals elicited production of antibodies specific for HIV-1 gp120/Env due to the induced antigen-mimicking ability. Neutralization potential of VRA proteins-induced serum antibodies gives an evidence of perspective of this unique mimicking proteins for the development of preventing vaccine against HIV and for the induction of protection against development of AIDS.

TABLE 2
Neutralization of HIV-1 pseudoviruses by sera of mice immunized in experiment II.

| | Reciprocal serum dilution resulted in 50% neutralization of pseudoviruses | | | | | | |
|---|---|---|---|---|---|---|---|
| | | | | | | VRA177 + | |
| | | | preimune | | | VRA017S + | |
| | Pseudovirus | Tier | (naive) | ABDwt | VRA177 | VRA017S | VRA017 |
| HIV-1 | pWITO | 2B | <30 | <30 | <30 | <30 | <30 |
| Clade B | pREJO | 2B | <30 | <30 | 31 | 61 | <30 |
| | AC10.0 | 2B | <30 | <30 | <30 | 48 | <30 |
| | TRO | 2B | <30 | <30 | <30 | 37 | <30 |
| | SC | 2B | <30 | <30 | 57 | 71 | 34 |
| | 422664 | 2B | <30 | <30 | 84 | 90 | 40 |
| | pRHPA | 2B | <30 | <30 | <30 | <30 | <30 |
| | PVO | 3B | <30 | <30 | <30 | <30 | <30 |
| HIV-1 | Du422 | 2C | <30 | <30 | <30 | <30 | <30 |
| Clade C | Du172 | 2C | <30 | <30 | 34 | 65 | <30 |
| | Du156 | 2C | <30 | <30 | 32 | 87 | <30 |
| | ZM53M | 2C | <30 | <30 | <30 | 32 | <30 |
| | ZM214 | 2C | <30 | <30 | <30 | <30 | <30 |
| | CAP210.2 | 2C | <30 | <30 | <30 | <30 | <30 |

Results are expressed as the reciprocal serum dilution which resulted in 50% virus neutralization.

### Determination of Protein Stability Via Fluorescence-Based Thermal-Shift Assay

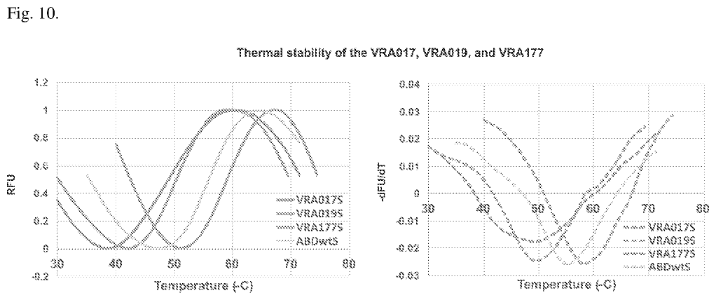

Protein samples (0.2 mg/mL) in PBS and 5× Sypro Orange dye (Sigma-Aldrich) were mixed in a total volume of 25 μL and measured using the real-time PCR Detection System CFX96 Touch (Bio-Rad Laboratories, Hercules, Calif.) as described previously (Kuchar M, Vankova L, Petrokova H, et al Human interleukin-23 receptor antagonists derived from an albumin-binding domain scaffold inhibit IL-23-dependent ex vivo expansion of IL-17-producing T-cells. Proteins 2014, 82: 975-89). Thermal stability of proteins VRA017S, VRA019S and VRA177S is shown in FIG. 10 with defined melting temperature (Tm) 50° C. for VRA017S and VRA019S and 58.5° C. in case of VRA177S. Melting temperature of parental non-mutated fusion protein His6-ABDwt-TolS-AVI is 55.5° C. (dotted line). The results show that amino acid substitution in randomized positions of ABD domain did not disrupt temperature stability of protein variants significantly. These suggest VRA-TolS proteins can be used as components in experimental vaccine development.

### Statistics

Differences between groups and statistical significance were determined by analysis of variance (ANOVA), Kruskal-Wallis test and Dunn's post-test. All statistical analyses were performed using SPSS v. 21 statistical packages (IBM Corp., Armonk, N.Y.) or GraphPad Prism 5 Software (GraphPad Software Inc, San Diego, Calif.).

1: A polypeptide mimicking an epitope of glycoprotein gp120 of HIV-1 which is recognized by a paratope of broadly neutralizing antibody VRC01, characterized in that the said polypeptide has the length up to 100 amino acid residues and contains an amino acid sequence:
(SEQ ID NO. 1)
X¹YKNX²INX³AX⁴X⁵VX⁶X⁷VKRX⁸IDX⁹ILAX¹⁰LP,
in which:
X¹ is selected from amino acids A, N, R;
X² is selected from amino acids A, R, D;
X³ is selected from amino acids R, V, P;
X⁴ is selected from amino acids V, L, S;
X⁵ is selected from amino acids T, G, R;
X⁶ is selected from amino acids G, T;
X⁷ is selected from amino acids L, A;
X⁸ is selected from amino acids V, I;
X⁹ is selected from amino acids G, A, R;
X¹⁰ is selected from amino acids R, A, G;
wherein an alpha-helical structure is C-terminally or N-terminally linked to said sequence SEQ ID NO. 1.

2: The polypeptide according to claim 1, characterized in that the amino acid sequence SEQ ID NO. 1 is selected from the group comprising:
(SEQ. ID NO. 3)
AYKNAINRAVTVGLVKRVIDGILARLP,
(SEQ. ID NO. 4)
NYKNRINVALGGTAVKRIIDAILAALP,
(SEQ. ID NO. 5)
RYKNDINPASRVGAVKRVIDRILAGLP,
wherein an alpha-helical structure is C-terminally or N-terminally linked to said sequence SEQ ID NO. 1.

3: The polypeptide according to claim 1, characterized in that the alpha-helical structure is sequence LAEAKVLANRELDKYGVSD (SEQ ID NO. 2).

4: The polypeptide according to claim 3, characterized in that it contains sequence selected from the group comprising:
(SEQ. ID NO. 6)
LAEAKVLANRELDKYGVSDAYKNAINRAVTVGLVKRVIDGILARLP,
(SEQ. ID NO. 7)
LAEAKVLANRELDKYGVSDNYKNRINVALGGTAVKRIIDAILAALP,
(SEQ. ID NO. 8)
LAEAKVLANRELDKYGVSDRYKNDINPASRVGAVKRVIDRILAGLP.

5: The polypeptide according to claim 1, characterized in that it contains affinity purification and/or detection tag.

6: A chimeric protein, characterized in that it contains
the polypeptide according to claim 1, and
a helical protein TolA, or its truncated version TolS, or serum albumin or heat shock protein hsp70.

7: A DNA sequence, characterized in that it is selected from the group comprising a complementary DNA coding for the amino acid sequence of the polypeptides according to claim 1, and a DNA hybridizing with said complementary DNA under conditions of high stringency.

8: A method of preparation of polypeptides or recombinant proteins produced in bacterial, yeast, insect, mammal or human host cells, comprising the step of providing the DNA sequence according to claim 7.

9: A host cell, characterized in that it contains at least one DNA sequence of claim 7.

10: A method of prevention of HIV-1 virus infection, comprising the step of providing a medicine comprising the polypeptide according to claim 1 as an active ingredient or an auxiliary ingredient of a vaccine to a subject in need thereof.

11: A method of preventing from HIV-1 virus infection comprising the step of providing the polypeptide according to claim 1 as an antigen for stimulation of neutralizing antibodies suitable for development of a vaccine to a subject in need thereof.

12: A method of prevention of HIV-1 virus infection comprising the step of providing a DNA coding for polypeptide according to claim 1 as an active ingredient of a DNA vaccine to a subject in need thereof.
